(19) United States
(12) Patent Application Publication
(10) Pub. No.: US 2023/0078419 A1
(43) Pub. Date: Mar. 16, 2023

(54) METHOD FOR DETERMINING MALE INFERTILITY ASSOCIATED WITH ABNORMAL EXPRESSION OF SEX-DETERMINING GENE ON X CHROMOSOME (SDX) OR SDX PROTEIN

(71) Applicant: Wuhan University, Wuhan (CN)

(72) Inventors: Mengcheng LUO, Wuhan (CN); Peng CUI, Wuhan (CN); Yuerong WU, Wuhan (CN)

(21) Appl. No.: 17/816,733

(22) Filed: Aug. 2, 2022

(30) Foreign Application Priority Data
Aug. 2, 2021 (CN) 202110879098.2

(51) Int. Cl. C12Q 1/6883; G01N 33/68

(52) U.S. Cl. CPC C12Q 1/6883; G01N 33/689; C12Q 1/6806

(57) ABSTRACT

A method for determining male infertility includes detecting abnormal expression of sex-determining gene on X chromosome (SDX) or SDX protein using a test kit.

### CROSS-REFERENCE TO RELAYED APPLICATIONS

Pursuant to 35 U.S.C. § 119 and the Paris Convention Treaty, this application claims foreign priority to Chinese Patent Application No. 202110879098.2 filed Aug. 2, 2021, the contents of which, including any intervening amendments thereto, are incorporated herein by reference. Inquiries from the public to applicants or assignees concerning this document or the related applications should be directed to: Matthias Scholl P C., Attn.: Dr. Matthias Scholl Esq., 245 First Street, 18th Floor, Cambridge, Mass. 02142.

### BACKGROUND

The disclosure relates to the field of biological detection, and more particularly, to a method for determining male infertility associated with abnormal expression of sex-determining gene on X chromosome (SDX) or SDX protein using a test kit. Sex-Determining gene on X chromosome, also known as SDX (9430086K21Rik, PWWP3B, or MUM1L1), is a gene for regulating fetal sexual differentiation and adult spermatogenesis.

Male infertility is associated with a variety of genetic disorders. For example, 25 percent of non-obstructive azoospermia is caused by chromosomal abnormalities or gene mutations. Identification of causative genes helps to elucidate the underlying causes of male infertility and makes it easier to select therapeutic drugs, thereby increasing the male fertility rate. Disorders of sex development (DSD) are mainly characterized by atypical external genitalia, abnormal gonadal development, with or without the presence of Müllerian structures. DSD are classified, depending on karyotype, into the following three categories: 46, XX DSD, 46, XY DSD, and sex chromosome DSD. 46, XY DSD patients with abnormal gonadal development lead to the development of the ovaries or ovotestis, which is manifested by pure gonadal dysgenesis. The patients with 46, XY DSD refer to 46, XY females that present internal and external genitalia and appear partially or even fully feminine, which are mostly caused by complex gene mutations, with an incidence rate of about 1/6000. Since the 1990s, researches confirmed that mutations of SRY (Sex-determining Region on the Y chromosome) cause complete male-to-female sex reversal (XY female) in humans. Although researchers have uncovered less than ten gene mutations associated with XY female, more genetic etiology and pathogenesis of the diseases are still to be discovered.

### SUMMARY

The disclosure provides a method for determining male infertility associated with abnormal expression of sex-determining gene on X chromosome (SDX) or SDX protein using a test kit. The method aims to detect the abnormal expression of SDX gene or SDX protein that causes male infertility, such as complete male-to-female sex reversal (XY female), seminal vesicle agenesis, and age-related male infertility. The expression pattern of the SDX gene or the SDX protein encoded by the SDX gene is analyzed and used to elucidate a molecular mechanism responsible for male infertility.

The method for determining male infertility comprises detecting abnormal expression of sex-determining gene on X chromosome (SDX) or SDX protein using a test kit.

In a class of this embodiment, the term “male infertility” refers to 46, XY DSD, seminal vesicle agenesis, age-related male infertility, or a combination thereof.

In a class of this embodiment, the term “abnormal expression of the SDX gene” refers to mutation in a promoter or enhancer of the SDX gene, deletion, insertion or substitution of one or more bases in a coding sequence of the SDX gene, or a combination thereof.

In a class of this embodiment, the term “abnormal expression of the SDX protein” refers to a decrease in the SDX protein expression, premature termination of the SDX protein synthesis, absence of the SDX expression, deletion, insertion or substitution of amino acids in a functional domain of the SDX protein, or a combination thereof.

In a class of this embodiment, the term “test kit” refers to a kit for detecting an expression level of the SDX gene or SDX protein in a male patient.

In a class of this embodiment, the test kit for detecting the expression level of the SDX gene comprises a plurality of reagents for whole genome sequencing.

In a class of this embodiment, the test kit for detecting the expression level of the SDX protein comprises an antibody specifically binding to the SDX protein.

The disclosure further provides a method for determining male infertility comprising applying a test kit comprising an antibody specifically binding to a SDX protein.

The following advantages are associated with the method for determining male infertility of the disclosure:

The method for determining male infertility detects the abnormal expression of the SDX gene or the SDX protein that causes male infertility, such as complete male-to-female sex reversal (XY female), seminal vesicle agenesis, and age-related male infertility. The expression level of the SDX gene or the SDX protein is measured and analyzed to elucidate the molecular mechanism responsible for male infertility.

### DETAILED DESCRIPTION OF THE DRAWINGS

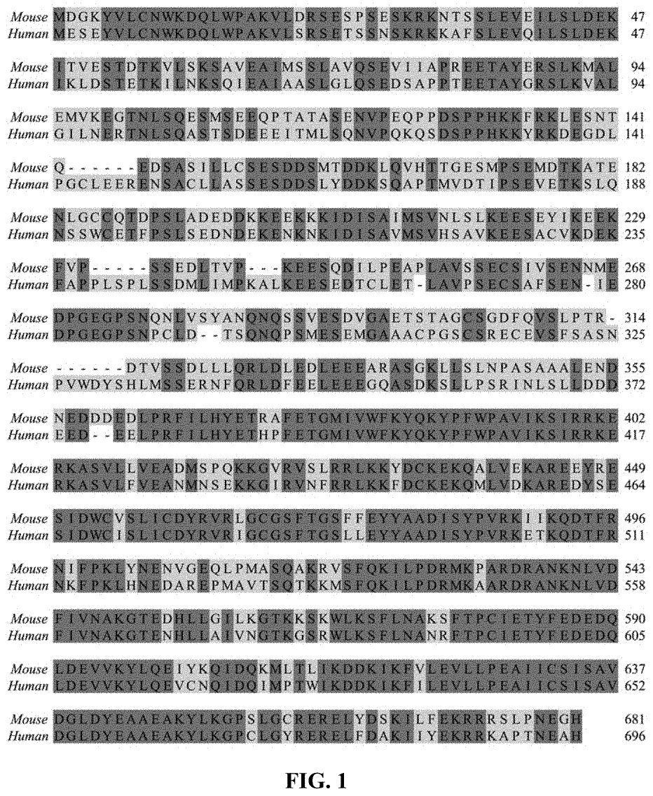

FIG. 1 shows an alignment of sequence conservation of SDX proteins in humans and mice;

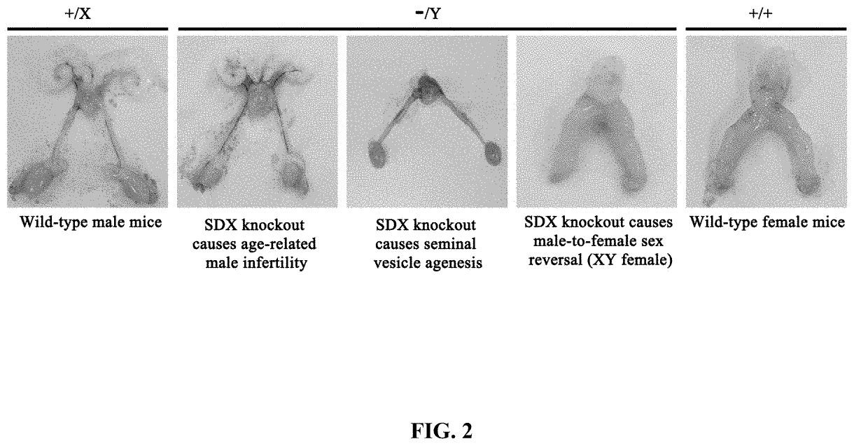

FIG. 2 is an image of SDX knockout mice characterized by complete male-to-female sex reversal (XY female), seminal vesicle agenesis, and age-related male infertility; and

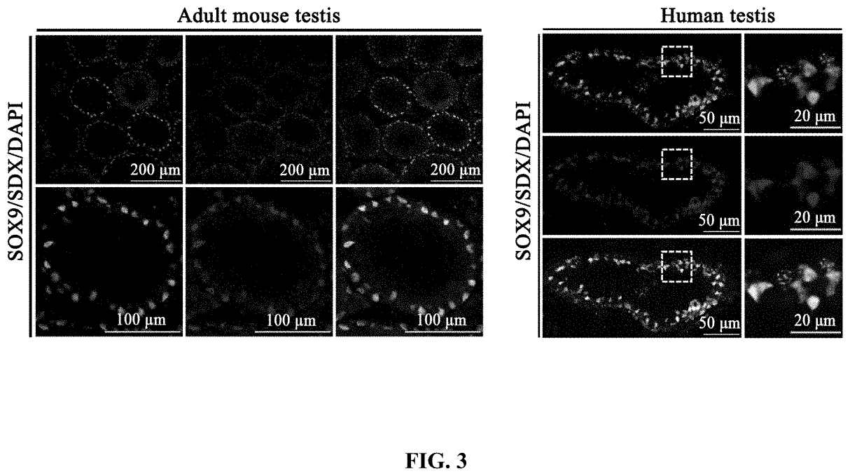

FIG. 3 is an image of localization of SDX proteins in humans and mice.

### DETAILED DESCRIPTION OF THE EMBODIMENTS

Unless otherwise specified, raw materials, reagents, and equipment described in the disclosure are available on the market.

In a proteomic study on the impact of genetic factors (gene mutations) on disorders of sex development (DSD), researchers found that an SDX abnormality may cause C57/6J male mouse sex to reverse into an XY female, or may result in seminal vesicle agenesis in SDX knockout male mice. The size difference in testes between wild-type male mice and the SDX knockout male mice increases with age. The SDX knockout male mice have defects in spermatogenesis, resulting in male infertility; and the SDX knockout male mice is fertile.

Further, the absence of SDX leads to defects in XY mouse embryonic sex differentiation period, with the potential for bisexual differentiation gonadal to male gonadal differentiation.

Further, the SDX proteins in humans and mice perform the same function because they share a high degree of sequence conservation and have identical localization patterns.

Further, recent research shows that abnormal expression of the SDX gene or the SDX protein causes complete male-to-female sex reversal (XY female), seminal vesicle agenesis, and age-related male infertility.

An expression pattern of the SDX gene or the SDX protein is analyzed to elucidate the molecular mechanism responsible for male infertility.

The abnormal expression of SDX gene is detected by whole genome sequencing; and the abnormal expression of SDX protein is detected by using an antibody specifically binding to the SDX protein. Therefore, it can be prepared as a male infertility test kit.

As used herein, the term “SDX gene” refers to a gene (MUM1L1; PWWP3B) located on chromosome Xq22.3 in humans, identified with an accession number NC 000023.11 (106168278.106208961), and contains six exons. The SDX gene has two transcripts (NM 001171020.2 and NM 152423.5), both of which encode the same protein that is highly expressed in reproductive system. Previously, the structures and functions of SDX gene and SDX protein are unclear.

The sequence of the SDX gene in humans is shown in SEQ ID NO: 1;

The coding sequence of the SDX gene in humans is shown in SEQ ID NO: 2; and

The sequence of the SDX protein in humans is shown in SEQ ID NO: 3.

To further illustrate the disclosure, embodiments detailing the method for determining male infertility associated with abnormal expression of sex-determining gene on X chromosome are described below. It should be noted that the following embodiments are intended to describe and not to limit the disclosure.

### EXAMPLE 1

1. Construction of SDX Knockout Mouse Model

Gene knockout method: CRISPR/Cas9 technology was used to knockout a gene of interest via homologous recombination. The gene knockout method was performed as follows: guide RNAs (gRNA) were designed and synthesized by in vitro transcription.

(as shown in SEQ ID NO: 4)
gRNA1: ACCCCCACATATGATCCTCA;
and
(as shown in SEQ ID NO: 5)
gRNA2: CCATTTGATGACCTATTCAA.

Cas9 and gRNAs were injected into mouse zygotes. Cas9 protein was directed by gRNAs to a specific genomic location so as to cut both strands of DNA, thus generating double-strand breaks (DSBs). And cells launched an emergency response to repair the DBSs via non-homologous DNA end joining (NHEJ), which results in a genetic deletion of the gene at the specific genomic location, thus knocking out the gene of interest.

2. C57/6J mouse was euthanized and then dissected; and reproductive system was isolated from the C57/6J mouse and observed.

As shown in FIG. 2, the SDX knockout mice presented male infertility, such as complete male-to-female sex reversal (XY female), seminal vesicle agenesis, and age-related male infertility.

3. As shown in FIG. 1, the SDX proteins in humans and mice performed the same function because they shared a high degree of sequence conservation and had identical localization patterns.

### EXAMPLE 2

1. Localization of SDX proteins in testicular tissues of mice and humans.

The testicular tissues of adult male mice and humans were respectively taken, fixed, dehydrated, embedded in NEG-50 medium (Thermo Fisher Scientific) at −80° C., frozen, and sectioned. First, the frozen section tissues were then blocked, then incubated with a primary antibody, washed, finally incubated with a secondary antibody, washed, covered with a coverslip, and observed under a fluorescence microscope; and fluorescence images were captured.

2. Result Analysis

As shown in FIG. 3, the SDX proteins in testicular tissue of adult mice and humans were both located in Sertoli cells (50X9 positive cells).

The results showed that the SDX proteins in humans and mice performed the same function because they shared a high degree of sequence conservation and had identical localization patterns. Understandably, an abnormal expression of SDX gene or SDX protein in humans is associated with male infertility, such as complete male-to-female sex reversal (XY female), seminal vesicle agenesis, and age-related male infertility.

Subsequently, peripheral blood samples were collected from patients with complete male-to-female sex reversal (XY female), seminal vesicle agenesis, as well as age-related male infertility, respectively; and Examples 3 and 4 were implemented to detect the abnormal expression of SDX gene or SDX protein in the samples of peripheral blood from the patients.

### Example 3

Determination of SDX gene sequence in genome

1. Materials

200 μL of each of the peripheral blood samples was taken.

2. Extraction of genomic DNA from the peripheral blood samples

A blood DNA extraction kit (Cat. No. DP304, TIANGEN) was used to extract genomic DNA from the peripheral blood samples according to the manufacturers' instructions listed below:

(1) each of the peripheral blood samples was treated (if a volume of each peripheral blood sample was 200 μL, the step 1) was skipped);

(2) 20 μL of Proteinase K solution was added and mixed thoroughly.

200 μL of GB buffer was added, mix by inversion, and allowed to stand at 70° C. for 10 minutes to become a clear solution; the clear solution was centrifuged so that water droplets were removed from an inner wall of a cap of a centrifuge tube;

(3) 200 μL of absolute ethanol was added and shaken for 15 seconds, during which time a flocculent precipitate may appear; and the resulting mixture was centrifuged so that water droplets were removed from an inner wall of a cap of a centrifuge tube;

(4) the resulting mixture in step 3) that contained the flocculent precipitate was added to an adsorption column CB3 (placed in a collection tube) and centrifuged at 12,000 rpm for 30 seconds; a supernatant was decanted; and the adsorption column CB3 was placed back into the collection tube;

(5) 500 μL of a GD buffer (mixed with absolute ethanol before use) was added to the adsorption column CB3, centrifuged at 12,000 rpm for 30 seconds; a supernatant was decanted; and the adsorption column CB3 was placed back into the collection tube;

(6) 600 μL of a rinsing solution PW (mixed with absolute ethanol before use) was added to the adsorption column CB3, centrifuged at 12,000 rpm for 30 seconds; a supernatant was decanted; and the adsorption column CB3 was placed back into the collection tube;

(7) repeating step 6);

(8) the adsorption column CB3 was placed back into the collection tube, centrifuged at 12,000 rpm for 2 minutes; a supernatant was decanted; the adsorption column CB3 was allowed to stand at room temperature for several minutes, and the rinsing solution PW remaining in the adsorption material was dried;

(9) the adsorption column CB3 was placed into a clean centrifuge tube; 50-200 μL of a TE buffer was added to a middle part of an adsorption membrane of the adsorption column CB3, allowed to stand at room temperature for 2-5 minutes, and centrifuged at 12,000 rpm for 2 minutes, so that a DNA solution was transferred into the clear centrifuge tube; and

(10) the DNA solution in step 9) was stored at −20° C. or −80° C.

3. The DNA solution was sent to a genome sequencing company for whole genome sequencing.

4. Analysis of the SDX gene sequence in genomic DNA

(1) If no mutations occurred in the exon sequence of the SDX gene, the SDX gene was not associated with complete male-to-female sex reversal (XY female), seminal vesicle agenesis, and age-related male infertility.

(2) Mutation of the sequence of the promoter or enhancer in the SDX gene:

when mutations occurred in the sequence of the promoter or enhancer but the SDX protein expressed normally, the SDX gene was not associated with complete male-to-female sex reversal (XY female), seminal vesicle agenesis, and age-related male infertility; and

when mutations occurred in the sequence of the promoter or enhancer and caused a decrease in the expression level of the SDX protein, the SDX gene was associated with complete male-to-female sex reversal (XY female), seminal vesicle agenesis, and age-related male infertility.

(3) Insertion or deletion of bases in a coding sequence of the SDX gene:

when a deletion or insertion where size was a multiple of 3 bp occurred in the exon sequence of the SDX gene, the nature of the SDX protein was changed drastically, illustrating that the SDX gene was associated with complete male-to-female sex reversal (XY female), seminal vesicle agenesis, and age-related male infertility; and

when a deletion or insertion where size was a multiple of 3 bp occurred in the exon sequence of the SDX gene, one or more amino acid residues was deleted from or inserted into a functional domain of the SDX protein, causing a decrease in biological activity of the SDX gene; it is necessary to further determine the SDX protein sequence by analyzing expression pattern and functional verification.

(4) Substitution of bases in a coding sequence of the SDX gene:

when no synonymous mutations occurred in the exon sequence of the SDX gene (that is, a change in the exon sequence did not alter the amino acid it generated), the SDX gene was not associated with complete male-to-female sex reversal (XY female), seminal vesicle agenesis, and age-related male infertility;

when a synonymous mutation occurred in the exon sequence of the SDX gene (that is, a change in the exon sequence altered the amino acid it generated), the SDX gene was associated with complete male-to-female sex reversal (XY female), seminal vesicle agenesis, and age-related male infertility.

(5) When the patients carried other known disease-related genetic mutations within human genome, the above diseases may be caused by polygenic variants.

Example 4

Detection of expression level of the SDX protein

1. Tissue sampling

Fine-needle aspiration was performed to collect a reproductive tissue sample from the patients with complete male-to-female sex reversal (XY female), seminal vesicle agenesis, and age-related male infertility.

2. Preparation of protein samples

(1) A protease inhibitor cocktail (Cat. No. 11836145001, Roche) was added to 100 μL RIPA Lysis Buffer (Cat. No. P0013K, Beyotime) to yield a final concentration of 1X; the reproductive tissue sample in the step 1) was added, lysed, ground on ice for 40 times, shaken at 4° C. for 30 minutes, centrifuged at 12,000 g at 4° C. for 10 minutes; and the supernatant was collected and acted as a protein sample for subsequent experiments;

(2) the concentration of the protein sample was measured by a BCA protein assay kit (cat. P0012, Beyotime); and

(3) 5X SDS-PAGE protein loading buffer (cat. RM00001, Abclonal) was added, mixed, heated in a boiling water bath for 10 minutes, and cooled in an ice bath.

3. Western blotting

(1) A gel casting apparatus was prepared according to the manufacturers' instructions and used to prepare a gel that contained a 5% stacking gel poured on the top of a 12% separating gel;

(2) Sample loading: 10-100 ng of the cooled protein sample was load onto the gel.

(3) Electrophoresis: a power supply was connected correctly to a positive electrode and a negative electrode of an electrophoresis tank; the protein sample run through the stacking gel at a constant voltage of 80 V, and through the separating gel at a constant voltage of 120 V. When bromophenol blue front reached the bottom of the gel, the electrophoresis was stopped.

(4) Membrane transfer: the gel was removed from a glass plate; a porous pad, a filter paper, the gel, PVDF membrane, three filter papers, and a porous pad were disposed successively to form a sandwich structure of layers, transferred into a transfer tank, cooled in an ice bath, and run at a constant current of 320 mA for 100 minutes.

(5) Blocking: the PVDF membrane was removed from the sandwich structure of layers and transferred into an antibody incubation tank; and 5% skim milk in TBST (weight to volume) was added to block the PVDF membrane at room temperature for 1 hour.

(6) Primary antibody incubation: a rabbit anti-human SDX polyclonal antibody as a primary antibody was diluted with 3% bovine serum albumin (BSA) in TBST (weight to volume) in a ratio of (1:100)-(1:200); and a GAPDH rabbit monoclonal antibody (Cat. No. A19056, Abclonal) as a primary antibody was diluted at 1: 500-1: 2000. Each primary antibody was incubated overnight at 4° C.

(7) Washing: TBST Buffer was added four times, 5 minutes each time, to wash away non-specific binding on the PVDF membrane.

(8) Secondary antibody incubation: a goat anti-rabbit IgG (H+L) HRP (Cat. No. AS014, Abclonal) as a secondary antibody was diluted with a TBST Buffer in a ratio of 1:5000 and incubated at room temperature for 1 hour.

(9) Washing: TBST Buffer was added four times, 5 minutes each time, to wash away non-specific binding on the PVDF membrane.

(10) Enhanced Chemiluminescence: a developer solution (Cat. No. 1863094, Thermo Fisher Scientific) was mixed with solutions A and B in the darkroom, and added drop-wise onto the PVDF membrane.

4. Data calculation

The BCA protein assay kit was used to determine the total level of protein in a solution; the level of GAPDH protein is constant in the cells because it is not affected by the change of a target protein, and GAPDH protein is thus used as an internal reference protein for normalizing the level of the SDX protein (a ratio of the SDX protein level to the internal reference protein level was taken as a measurement value); and the SDX proteins in control groups were extracted from normal testis and ovary tissues. The developed images of different samples were processed in grayscale and the grayscale value or grayscale area was calculated.

5. Result analysis

No differences (in the position and size of SDX protein bands) between the experimental groups and the control groups: the SDX protein was expressed at the same level in the normal human cells and the patient cells; and when no mutations occurred in the SDX gene, the SDX gene was not associated with complete male-to-female sex reversal (XY female), seminal vesicle agenesis, as well as age-related male infertility.

A decrease in the fluorescence of the SDX proteins in the experimental groups: the sequence variation of the SDX gene were determined to analyze whether the SDX gene was associated with complete male-to-female sex reversal (XY female), seminal vesicle agenesis, and age-related male infertility.

A change in position of the SDX protein bands in the experimental groups: the results showed that the termination of the SDX protein happened prematurely or a new sequence was inserted due to an absence of a stop codon caused by insertion of one or more bases; when the expression level of the SDX protein remained unchanged, the SDX protein performed normal biological functions; when the expression level of the SDX protein increased, the SDX protein function may be reduced and was validated using protein-protein interaction methods (CO-IP or GST poll-down).

An increase in the content of the SDX protein in the experimental groups: the results showed that the biological activity of SDX protein was decreased; when the SDX gene sequence alignment and protein-protein interaction were determined to accurately analyze whether the SDX gene was associated with complete male-to-female sex reversal (XY female), seminal vesicle agenesis, and age-related male infertility.

6. Tissue fixation and dehydration

The tissues collected through fine-needle aspiration was fixed with 4% PFA on PBS (weight to volume) at 4° C. for 2 hours, and then dehydrated with 30% sucrose in PBS (weight to volume) overnight at 4° C.

7. Tissue embedding and sectioning

The dehydrated tissues were embedded in NEG-50 medium (Thermo Fisher Scientific) at −80° C., frozen, and sectioned.

8. Fluorescent immunohistochemistry of frozen tissue

Blocking: a Pap pen is used to draw a circle around the section; the section was completely covered with 10% goat serum in TBS (v/v) as a blocking solution, placed in a humid chamber, and incubated at room temperature for 1 hour.

Primary antibody incubation: after removal of the blocking solution, the primary antibody (that is, a rabbit anti-human SDX polyclonal antibody was diluted with 10% goat serum in TBS in a ratio of 1:50) was added drop-wise onto the section; the section was completely covered with the primary antibody, placed in the humid box, and allowed the primary antibody to incubate overnight at room temperature.

The section was washed once with TBST for 5 minutes and washed with TBS for three times, each 5 minutes.

Secondary antibody incubation: anti-alexa fluor 488-conjugated goat anti-rabbit IgG (H+L) antibody (cat. AS053, Abclonal) was diluted with 10% goat serum in TBS to form a secondary antibody; the section was completely covered with the secondary antibody, placed in the darkroom, and allowed the secondary antibody to incubate at room temperature for 1 hour.

The section was washed once with TBST for 5 minutes and washed with TBS for three times, each 5 minutes.

Nuclear staining: mounting medium with DAPI was dropped on the section; then the section was covered with a coverslip and observed under a fluorescence microscope; and fluorescence images was captured.

9. Result analysis

The SDX proteins in the experimental groups had no fluorescence: the SDX gene sequence and results of the western blotting depicted that the termination of the SDX protein happened prematurely or the SDX protein was not expressed, causing complete male-to-female sex reversal (XY female), seminal vesicle agenesis, and age-related male infertility.

The cells in the control groups had no fluorescence as well.

No mutations in the exon sequence of the SDX gene: when the SDX gene sequence and the results of the western blotting showed no differences in the size and content of SDX protein between the experimental groups and the control groups, the SDX gene was thus not associated with complete male-to-female sex reversal (XY female), seminal vesicle agenesis, and age-related male infertility.

A deletion or insertion where the size was a multiple of 3 bp occurred in the exon sequence of the SDX gene: when the position of the SDX protein band in western blotting was changed, the nature of the SDX protein was changed drastically, illustrating that the SDX gene was associated with complete male-to-female sex reversal (XY female), seminal vesicle agenesis, and age-related male infertility. It is necessary to verify the coding sequence and the function of the SDX protein.

A decrease in the fluorescence of the SDX protein in the experimental groups: the sequence mutation of SDX gene and the expression pattern of the SDX protein in western blotting were determined to accurately analyze whether the SDX gene was associated with complete male-to-female sex reversal (XY female), seminal vesicle agenesis, and age-related male infertility.

The results of Examples 3 and 4 showed that the abnormal expression of SDX gene or SDX protein occurred in the samples of the peripheral blood from the patients with complete male-to-female sex reversal (XY female), seminal vesicle agenesis, as well as age-related male infertility.

It will be obvious to those skilled in the art that changes and modifications may be made, and therefore, the aim in the appended claims is to cover all such changes and modifications.

What is claimed is:

1. A method for determining male infertility, the method comprising detecting abnormal expression of sex-determining gene on X chromosome (SDX) or SDX protein using a test kit.

2. The method of claim 1, wherein the term “male infertility” refers to 46, XX disorders of sex development (DSD), seminal vesicle agenesis, age-related male infertility, or a combination thereof.

3. The method of claim 1, wherein the term “abnormal expression of SDX gene” refers to mutation in a promoter or enhancer of the SDX gene, deletion, insertion or substitution of one or more bases in a coding sequence of the SDX gene, or a combination thereof.

4. The method of claim 1, wherein the term “abnormal expression of SDX protein” refers to a decrease in the SDX protein expression, premature termination of the SDX protein synthesis, absence of the SDX expression, deletion, insertion or substitution of amino acids in a functional domain of the SDX protein, or a combination thereof.

5. The method of claim 1, wherein the term “test kit” refers to a kit for detecting an expression level of the SDX gene or SDX protein in a male patient.

6. The method of claim 5, wherein the test kit for detecting the expression level of the SDX gene comprises a plurality of reagents for whole genome sequencing.

7. The method of claim 5, wherein the test kit for detecting the expression level of the SDX protein comprises an antibody specifically binding to the SDX protein.

8. A method for determining male infertility comprising applying a test kit comprising an antibody specifically binding to a SDX protein.


SEQUENCE LISTING (5 sequences)

SEQ ID NO: 1 - DNA, 40684 bp, synthetic construct
  source 1..40684: mol_type = other DNA
gagcttggaagaaagctgcgcccagccagtcttcggggaggagctgcattacacacaggcttcggaggcttccgtggagaagcttggaggtaatggctgtcgtaaatgctagttattttcagagagttctcgaagatttggaataaaggtgagaaaagaagagagtgctgcttaggaataacgggaaaggagtgtcgttctgtgagggtcgttgtttctgtgagggaattaatatctgtgttttgtgttccttcagattattttaaaagtccacagtcggggaggtggtaatcaacattacacctgtggaaaaatattcttaaagaggtagcaaaagctaactttttaatagagcattttatgtttccatcaaaaggcagtggtggctgagataaatgatgaaacaaaacttgggagtttaatcttccttattttgaaaaaggttgtaatagtcttaaatgaaaaattaagttgttggggatgttggttttttatattactttgaatgtgttgtgtttctaacaaatcttactgttttggtggagtatgttagtaatttaaagtaatgcataactgtgcaaattcgagtatctctgagaattagtttggttatttgtcaagggatacacaatgacttaatataatagctttttaaataatgtgcaaaatatgatctttgtctgtacttctacctatctctcaagagaattgtacattcctatttgtactgcaatggaaaaaatctaaaagatcataacaataacagacgtgcattagtataataaaacacaacaaacaatatacttagtaataaaggatgcctgataaaaaaggatgtcaggggaaaaaggaggaaattttccacttctttcttccacattttacttagcatgtagtttctatgtcggcatagaaactgagatgagaacttaaaatcatggggaaaagctactttccccaaaatagcgtaaactcatatataaacagggaaaggtgttcaaatatgtggctaaataaatgtaattttaaatagtatattcaaacatgtatgtgatgaggaaaaaaataaaaaggattagacatcaaatgtatgccgtgaacaactcagggtttgagtactgatgatgtgtattttcatctttagatttttatgtacttttagattcttttggaaacgataatggagctgaaaagggaataatcacttttgttacagacacttgagcaaatagccacacacacattccattgtctttagctgcagtaggatttttctatgggtctgagaaggctcattacctgattgcttgtgcttttcttgcaattcaactattgaggctatagtccagactctggttcaagcagaacatatttatctttatactcctgaggaataattcgtaaaatgttttaaacatattatgacatagtcatataggatagtatatctataatgttgaatactacgcagctgtaaaaggaatgagttaactctaaatgcaatgattagagtaaatgtccaaggtagtttctaaaaataagcaaattgaagaatggcctgtatcatgtaatcctatttgtttaccaaaaagaatgtgcatgcattcgtacagtgtatgcaataaaaacctggaaatatacatgctactttattaattggagaaatctggaaataatacatgccactttattaattggagatataagtgttcttatatctccacacatatctccaattaatagacagaatattttaaccttctatgtattacatttccccattttaaaaatttcattttgaaaagagcaagcctgaataattttcatcatgagaaaaacataaaattttagaaatgtggaaaaaattttgtaaagcaattcacaaaaagatgatatggaaaataattattgatatggaaagatgttcaaatatattactggaggaataaagggtctagtttcaaaaagcttccacattggtaagtctctacagaaaaaaatctagaaatatacagagcaaaccattacattttgaccattgttgtggagatttcctttcttctttggaaatccaaaagatgtagaaagatatatacaatttgcatgagagatattatgatgaaaaatattaaaatgagaacattatgatcccatttttgtataacacatgcacagaactcttaaactgagaatgaaaaattctagaagcctgagtgtctgcagttctggttctggtcacaagatggtgcttgccaggtttactaagctttgtccaggcaaatatttttatgaagtggacaaagcaatgaaatatgttttgtattagccgactatgagacagcatgtagagtgtgattgaaattccattcaaaatattataaatatctagaagaaaaatccccaccaataactgacatgaagactcacacagaaatcataaaaaatgctgctacatagagtctggcccatagtagaaacaagtcaattgttctttcccttctctcttttctccacctcataattttcctgaaggatatgattttagcatgaacgaaataacataactgacccagagagcatttcatattataataacatgaaggaaaatagatggcatctacatcacagatgagattttgcctcattcctgtgttttggaatgacagcctgttagagtttaggtagggaaatttatttacaccttttccatttcattggtttgattccctgccagccgagccccagaaagacaggtcaaccacagaagtgctgagccaggtaaagaccctgctggacaagcagctggagtgagaatcaagacagctggaccacaggaccagacccagcagtatccatgtaacagtgagtaagactcttcccatttcacaaaacatttcacggtataaacatgttgagaacttggagaacaatgtattttgcttccggctctacagtcatcatagcattttaaattaaaagccacatttcatgtaaggattatgttaggttgagaatttggggtgtacttcattttgtttcctgttctacagctatcatgtttgtaattaaaagtcataacttctagtttttggaaagaatccatatattataccactatgaagaaatcatgcaagaggctatgcaggtgtctccctgaggcctgactctgtagtccttgacaactatactaggttgtcataccataaactagataacatagttataaaggtttcttagaacctgcttaaggtcttatgaccttcttgaatcacctgtacagtcatgcatggcttaacgatggggacatattctgagaaatgcttcaataggcgattttatctgagaacattatagagtgtacttgcacgaacttagatggtatagcctactacacacttaggctatatggtatagcctactacacacttaggctatatggtatagcctattgctcctataccacaaacctgtataccatgttactgtactgaatactataagcagttgcaacacaactgcctacagtggtgaatactgaatactgtaggcagttgcaacacaactgcctacaacatggtgctgttgtctcctagaataacaatgcccttttctggaatacctcctgaaagacctgctctgtgcctattttacgattaacatttttttttctaagttgaaggaatatactaaaataagattaaaaagtatagtaagtacataaaccaataacagtcatttattatcattataatagtatttgtatatccaaacatatctaaatatagagaagttacagtaaaaatatgacataaaagacaaaaaatggtatacctgtataggggaattaccatgaatggagcttgcaggactggaagttactttgggtaagtcagtgagtgagtggtgagtgaaagtgaaggcctagaatattatgtatactactgtagcttgaacacttaggctacactaaatttataaaaaaaaatatttttttaataataaattaaccttagcttatggtaacattttactttataaatgtttaaatttttaaatcttttaacacttttgtaataataccttaaagcactaacacattgtacagcagtaaaaaaaataaaataaaataatttctttctttatatccttgttctataagcttttttcctattttaaaaaattttttattttttttcttttcaaacttttttttgttaaacaccaacacacatattagcctaggcctacacagggtcaagatcgtcaatatcaccatcctccatttacacatctcgtcccactggaaggtcttcaggggcagtaacactcatggtgctgttgtctcctagaataacaatgcccttttctagaataccccctgaatcaagacctgctctgtggctattttacggttagcattttttttttctaagtagaaggaatatactaaaataagattaaaaagtacagtaagtacataaaccaataacagtcatttattatcactatcaagtattaggtacgtcacataattgtatgtgctatacttttatgccactatcagtgtagtaggttgtttacaccagcagcaccacaaacaggtgagtcatgaattgtgcttcagtgttacaatggctgtgaccgacatcactaggcaacaggaattttgcagctttattataatcttacaggaacgtcgtcatatatgcagtttgtcattgactgaaatgtcactatgcacgacatgactatattctcttaaataatattttgtagtggtttaacaaacttaattctttcccagcaaccatcacaaatccgtgcctcataacgttgtgctgtcagtgagattgttgatgctgagcctcattgtgtccgtgagctctcctttggttgccttggatagacagggcagtagtggcagcttagaaaagattagaacccagcattcacttacgataactcagagggttgcaaaaggccaaattatccaagaacaaaaacaatgtccccgatactgccaaagctcctcccacacaattccttttttattacacaacagagaaaataacgatgggatattgggtttgtgttataatacttctcttttttgtacccccccaaatatattccacacatctaaccccacctatctgatacccattattcgttcttctgacttgtgtcttgtctatattctcatcgtctaccttcatcctacttagtaaagtaaattttttatctgatctcttttcttctttgccatgttttaatgcaatccaattatcacccattgaaaatactttattcaagctactttgtgaaggttcagggggataaaagaggctacaacagacgagaaccatttactatccccagataaatagaatgaggcttcttaaaaaacaaaaacaaacaaaaaacagcttttctagacaactaagtagctaaaactctttttattattgtatttttaaatatattaatggcttaatatttaatatttggacaatctaggttaatatagagatacaggtgacccatgcatccttgctgactatgctatctaggatatgggagatgacaacctagcattgttgtcaaggattggacagtttttctccgctgagctaataaaggtttcaaacccacactgtctgattttttggctgtctgaaaaattcagaaacataaccatgtcataatgccttggtgataaactcttacaagctaaacattgggtcctgccaacagaaatccatatctcaccaaagaactctgtgtcattttaaagttgcttaccaggaaaattttgacccaggctgacccttctgttatgagtctctggaagaatgatggcttctttctttagagtccccaacgcagacagttttcttttattccatttgatcattacaggaagagatgtgtcaaatgcagctctgtgtctctaacctgaatagcgagaatgtggcaggagatgtgtttacccactcactgtgataacaaatggggttgtgagggcctcaccaactttacctagttttatgattttttataacattatcttaagagacctaaacctcagaatcattatccattagcaatgtgtaggttctaatgcttcctgggaaacaacacctgaacattaaaggcaataaaaatctaaatttaaatcttaattttgtgaatattttgtgaataaaaaagctcagcaaaagaggataaaaatgcaactttcccatactgaattggcagctgcaaaccgcaaagcacaggagcaaggaattgccaattgttaatgagggtagaatgaaaatggcaattctctgtataacgaatgattttcagcccctgtccaggaatcaccatatctgaaagggaagctgctgccaggcccctataacacaaaagagacttctcataactgatctaaggccaagttaatgctatcatgcagatacggtcagtgcaaaagcatagtgcaggctgggagcggtggctcacaccttgtaatcgtagcattttgggaggccgaggcaggcagatcacctgaagtcaggagttttagaccaggctggccaacatggtgaaaccccatctctactaaaagtacaaaaattagctgggtgtggtggcacatgcctgtaatcccagctactcaggaggctgaggcaggagactcacttgaacccaggaggcagagtgagctgagatcgtgccactgcactacagcctgagcgacagagcaagactctgtctcaaaaaaaaaaaaaaaagaagaaaaaagcatagtgcatgaaaatactttttaatttgtgtgggaatgtagatggatgtaagagatagaaagaagtcaaggatagtaagcatggtagttgctgaagatggttgtagttcaccaatatgttcaccatcagagacatgagactaagcaacactcacgctggggttatttcttttttttttttttttttttttgagacggagtcttgctctgtcgcccaggctggagtgcactggcgcgatctcggctcactgcaagctccgcctcccgggttcacgccattctcctgcctcagcctcccgagtagctgggactacaggcgccagccaccgccccctgctgattttttgtatttttagtagagacggggtttcaccgtgttagccaggatggtctcgatctcctgacctcatgatctgcccacctcggcctcccaaagtgctggtattaccggtgtgagccaccgtgcccggccacgctggtgttatattcaccaatttcctcacagtttgaatgtgagcgacatcatctttggttctaccaaagaaacatgattccaagcccaggagggagtgaatagtggtggaaagagggcatacctcctgttttggagtcagactttggctccgattttgtctcaccctcttcctagctttgtgaccgtggcattagcaagagtgcccttgtttcctggatctctattagttgtatcatttatttgcttcttagtcaatttaagtagaaaaatgccatcttattgctgttttatatttgcatctctttgattattagggaaggtgaatatcttttatgtattagttttaatctttattttgaatatttgggttttgaggagtcccgatatatagcacctccctttctccactgaacaaactggatagatagatataaaacaaatggagttgcagatggaagccaaaatgtcaacattacaacagtacaagaggaagaggaagttgcggggtattgattattgtgagaatcaaatgggcttatatattaactattattttattttatttttttgagatggagtcttattctgttgcccaggctagagtgcagtggcgcgatctcagctcactgcaacctccacctcccgggttcaagtcattctcccacctcagccctctgattagctgggattacaggcacccaccaacacgcctggataatttttgtatttttagtagagatggggtttcatcatgttggccaggctggtctcgaaccgctggcctcaaatgatctgcccacctcggccttccaaagtgctgggattacaggcttgataacagaactccaaatttaagcaaacctaccaaaatagcctagaggtttgtagctgtacaggaagggacagagattcctgtggtggcaccatatgagcttgccctcaaggaagtaaatattaggagtggggaaaaattatactctcagaacattgtgaattgtctatttatgtcttttgcccatttttactctgccatttatttcttattaatatgtaagaactactttagtgatatttttgcgttgaaaacagttttgtcaattttgttcttatattttcaaatttggtttattgtgtttttgccaataagaatttttttgtttccaatttcaggatattcagatacctacatatctctttaaggattttggttgatgttttatgtttagaaatgactttagttgaaaaaaacgatgaaactttctgaaagatgaatgagaagacctgaataaaaagagagatatacatcatgtgccagggccagaagaattaaaatttcagggaattgcttatccacaaaataatgtattaattcattgcagtacaaataaggatgtagaggggctttttaatagagcttgaccagtcggatcttgaaatcgtttttagaaataagttgccaaagtaggcaagaaaaatatgtaaaagatgttctttactgagtctttattatatggtaaaggcatttctacctttgtttttcccagaaaatcacacactcttgtagtttgaatgtaaattaaacagacacatgttgccattccgctttcaactgctttaaaaatgatgatcttttcgctttaagggaattgcagtttaacttggtgatcctcttccctttgagggaaaggtccccttgctattcccatcactgggagggggcggagcctcgtcggagccgctcggaagggagctgtacccggccagtattgggggaggggcagcatttcaagaggctgcagctgacaagaagcttcaaggtaatggctgtcttaaagccagttattttctgggaatttcttcgggccttggtgttaaagtaaaaaaaaggaaagaaaagaaatagagattgttgcctgagagaggtgggaatggaatggttttgtttgaactgctgttattggaacggaattaatcctctgaatttttgtttcgcccgatatttttaaagtcctccgtgtgggaatgggcgatcaaattacagtttctaaattaagacttcggtgagacgtgctgttcgtgttaattttttacgtggcactacatatttctatctttgttacagattttattatattttgacttaaacacattattctacacaaaaaatgatagaaaaatgtaaacacacaaagagaatgacatatagggacacaaatataaatgtacatgaacatggaaagggattcagatatgccgtcgagtggtaacacttgtaaagatgtatattcgttttccttaggaagggaactagactcccgagttaacagtcagcatctctgagtgttcatctctgagtgttcagattatggaagtttttttaaaatctatttttcactacttccagcatttttagaattgtttctatttgtgcttgttttcataataagaagagcaaaataaagccaaaacagtatacccattttgtttcaaatacataaacacacatagacaccacagacacgctcccatactcccttgtcttttgtgaatagtggaatttttcctggacctgatcaagtgggtagcctggttgctgtgtcaactacagccttgcatgtgtcctccctgcagcccgctctttcagtccacaggtacagccatggtcagcaagaaccacttttctatatcatctaagtgtaagagtgagaaacggataggatttggtttatttctatgaacagttttaaacgtattatgtactatggtgcatccatacaatggcacgttatatagctgacaaatgaaagtatgaacttgtagtgacttgaaaaggtgtctgagaagtaaaaagagcaaactgcagaatggtaagcaccatattatgtcatttaaacataaaagactacattaatacatgtgtaaccaaatcagaaaaaatataaaccagcctgctaatggaattatttgtgcagttgaagagatagggcaggctttactatcactatttttgaaaaattcttaatacatatttagcctttttttaaaacaaaatcagaaaaaagattgaggtaaaatgttgcaaagttgggggaaaattgcatttgaagtgattcactaaaaaatcacattgaaaacacttagtagtatgcaaaaatgtttaaaccatgagcttaagcaggacaaaaagctgatttgaaataatatgcattcatacgtttatgtaaagaaaaaatatggaaagatatacaggaaatagtatctttccagaattatactttctttttatatatctttctcatattttcaattttcagtaaccatgcaattatttttctgcaatccgaccattggaggtgaagagattcggattccattttttacgtaatgcataacaaaactaaatttgaggatggtatattctagaagcctgagaaagctgcttgctggcaggatgtagttcccaccgttctcccacaaggatgttgtagtaacagttctgtttctcgtcacaagatggtgcttgcaagatatcctaaattttctccagacaactatttttatgaagtgagcaaacaaaacaatgaaatgtattttattatcagactatctgacagcatgtagagtgtgattcaaattccactcaaaaaattgtatatgtctaaaagaaaaaccccacccaagaactgatgtgaagcctcaaacagaaatcattaaaaatgctgctacatagtctgccccatagtagacacaagtcaattgtcctttcccttctctcctttctccaccttgtaattttcctgaatgatattattttagcatgagccaaataacataactgacaaagagagcatttcatattataagaacatgaagtgaaaatagatggcatctacatcacagatgagatttcccctcattcctgtgttttggaatgacatgcctgttaaagtttaggcagggaaatttatttacaccttttccatttcgttggtttgattctctgcctttatcagagatgcacttgacatgggaaatctgcatgtgatgccaggtgagcctctgagaaacagtggaaccacaaaagtgccaagcagggtaagggccctgccagacaagcagttggagtaagaattaacacagctggatcttagcgccagacccactactatctgtgtgacagtgggtaaggcttttcccatttcacaaagcatttcatggtaaaaacattctaagttgagaatatgggatacaactgatttcactctcgctcagtagccataacattttaaattaaaagccacagattctaatgttgggaaagaattatatatgctaaactcctgtaggaaaagtatgcaaacagtcataatgatgtctaattatggaagacttggatgccccagccatctgcctgactctcacgaaacccttaaaaacaatcctttccaatgggtggccgtggcagccattatgaatctccgacttctaagttttttctcttaagaaggtcgttgttgtctggattagtccaggggatgcagacccccagtatctgtgtctggctgccttgggtatacagggcagcaccgttagcttagaagagtctaaaaagaattcccctagaataagccttgagagactccaaagtacaaaaatattcattaataggaatagtgtccccaacatattccccagctttccctgggcatctctcaccaccctctcatacacacgtatattcattccttagatgtaataactataataagggctacaaactggggcattatagaactactccttttcttagatttttaaatatttcccccccaaccacaatatgtttatttgtcttgtgcccagtatttattcatcttttgtcttctccatttccattgtccatcttcctactcctttgagagaagaaatccgtttgcttattctcttttttctttgcctttcctttaaatgtataccctctagtattgcctatttgaaattacttattcaagcttccttgtgagttttcagggaggcatgaacaagctacagaagaagaaaactctttagtagccctaaggcaatagaggcctctaaaaccactagttttctggttaaaccagacaaacgcagaccagtgacctaagaatttttgttagatgattgaaagttcactgactgatttctaacctatatccagataataattcagatctatggtctatttgaaacctttttcctgtccccttcttttctatgtctatatcgacctcttgcttcacacttaaattgtcactgaactttaaaaaattattctaaccttgtaatatattttgatatctggtagcactagttgttcctccagtccactaataaagtcagaactaggtcctaaacccatgtctctatatctgtttatgaatttggaatacgtaaaaactttgccataatacttgcctgaggaactctgaggtttagatattggatcttcaacagtgtctcatatctccttactaaagaactgataagtacctttatttagagtggtggtgtgtccagagttggttccttccgatgggttcttggtctcgctgacttcaagaatgaagctgtggaccttcgcagtgagtgttacagctcttaaaggtggcacgggcccaaagagtgagcagcagcaagacttattgtgaagagtgaaagaataaagcttccacagcgtggaaggggacccgagtgggttgccactgctggctggggtggccagcttttattcccttatttgtccccgcccacgtcgtgctgattggtccattttacacagcgctgattggtccattttacagagtgctgattggtccattttacagagtgctgattggtgcgtttacaatcctttagctagacacagggggctgattggtgcatttttatagagtgctgattggtgcatttacaatcctctagctagacacagaacattgatttgtgcatttacaatcctctagctagacagaaaagttatccaagtccccactagacctaggaagtccagctggcttcacctctcaatccccgctctaaacaggacacccgaactgctgttaggaattgggcaatgaccgctctagctacttcctgctggataggggtgaagaaggggccctgcagttgtagtgtcctccagaggggaactctttaggccagtcaaagggccggcaggtcggtccatgggtcctcagtagaagttgctagttgagctcatttggggttccatttgtaagaccatctgtagcttgatggccttgatcctagaagaaataaatttgacaaggaggttaaaaatatagggcccaaaggcgagtaatagcaagatggctgtcacgggacctagaaaggggagaagccatgtcgcccaactccagaggttggtataagagtttgaaagttgttgtctgatttcagaagccttttcctgtaaacaccaggcagaatctcatactatccctgactggtcagtgtaaaaacaacactcttcccctaagaaggtgtagtcctcctttctcagcagtgaggaggtctaggccttggcggttttggagagtcactgctgccaaagagtctatttgggattgtagtgtaaggatagatttcattatttcttgcaaactatctgagaaatcctttgagagtgtgtggtagtaggataattaagtagataaactggctattccagttcctgtagcagtagtcattcctaaccctataagtaggggtattagttgtatggctctgtactgacggacttgagctttgaggggcactgatagggtctgatttcctggggcagtgttaatgttgggacttaggaagattaaagtgcaggtgcctgtccagttagtggggaggcagatataggttgatgttccacataagaagaatatgtcttagctgggtagacagaactggttgtgtatgttaaaaaggtgtgtgagtctgttgttttcattttcccatactaatagagtacttgccaaggtagctctggtgagtggctggaaaggggtgttgggagcaaactgagtggctccctgtgttctattttcccattggagaaaaaactaggaaccattcgagagggtgattgaaagaggggatgaaaagccattcactagtggtgggggcactgctgcaggggggtctggggtgaatggtcacgcagggagtatgtttgccattacaaaacctggactgtttgttagtaaaaggattccattaaaggggcaaggagcggtgttaaagataaagcttatgtaggttttcacttatcttttttaaggaggaaggggtttttcctcaggatcagcaggaggagcctttttagtctgggatgtttccttctgaaataggagacacaaatcctccaacggtttgcaggtgtatcaaggctggtctggcagatcctgggactcctgagctgacggtcctgcaggttcctcaggggatgtccaaagtttaactcgggtgtggtgaatccaagattccactcctgccacctaaactgcagtgggggtagagaggattactgagtatggtccttcccacaaagaatccatagatggggaggtagaggggagggatttgaccaacaatagatctcttggttgaagcaactccattcccttttctctgtgacatccttcaggtaggtttttgtactcctagaattggggtgttgcagggactactgcattttcttactaagccttgagcttttaaatgtctaacaatatcctgtaatactttatgagcttcaggtcttaagggatattgcttttgataaggaaaagtggtggggtcttttagcctgatttggactgggtgggcattttttgcccttccgaattgtccttccaatgcccagacttcaaggttgattccctcctcaaataggggacaacaaatgggtaacttgttccccatattcatgtagatcatagcttcagctttggctaatatgtccctccctaataagggtgtgggacttttaggcataagaaaggcatgtgaaaagagcaaagtctcccagttacaaatgaggaggtgggagaaatacctggttacaggctgtcccaggattccttggatggtaacgggccttgaggacagctgtctaggataggagataacactgagaaagacacgccagtgtccaggaggaagtcaatttcctggccctcaatggttaaacatatccggggctcagtgagggtgatgacatgagctggcgcttgtcctggtcaccctcagtcctgttgttggatcatctgtttggggcttctggcccagagaacctttgtcctttggggcagtgtgccttccagtgattgccttggcatagtggacatgggcgaggaggcagcttgtttcttgttggacaatcttttttaaggtgtctttgcaaacctcactggtaacaagccctaccgggtgattggcctgctccattttctgtcctctctgaaccaccaaggtttgtttgtctgaaggccatgactaaggctgcagcctttctctgatctcgcttttcctttttggcctgttcctcttggtccctattatagaacaccgaggttgccaggtttaataatgcctccagattttattcagggtccagggctcacttttggaggtttctcctgatacctggggctgattgggtaataaaattatctttttggatcagttgacccttgagggagttgggtgacaggggagtatattttcttaaggcctcccatagccgctggagaaaggcagaaggattttcttcctttccctgagttatggtggacatcattgagtaattcatgggctttttcctaattctccttagtccttctagaacacaggtcaacagatgtttgcaattccagtccccatgatctgagtcgaggtcccagtagggatccattaccggggatggcttgctgaccagtagggaatttgtccctttcttcagctgtcattctatcatttacttgactaagataccagatatcttcaaactcttgggctgcagctaaaactgcattgttttcattaaaggccagggtttgatctaacaatagcatgacatctctccaagtgagatcaaaggtttgctctaggctgtgtaggacatctatatacctatcgggattgtctgaaaacttccccaggtctgccttgatctgctttaaatcagagagggagaaggggacatgtacccaggttgggccaaattcccttccccctacagcttgaaggggacataaccgatagcccaggggtttttgtggtcccttggagatttctttgcttgcttccttctgggtgggggagattagaggaggcttatcattaataggaaggggagctgtagaaaggctagtatatggaggtaagctgagaggttctgctgtgggatgtaaattgcatgctttgcatagttgtggattctccttcaatgaaaagaaagcttgggcataaggtatttcactccatttgccctccctgttacagaaaaggtcaagctgcaggatagtattgtaatttatacttccctcaggtggccatttttccccatcagacagagaatattggggacaggccatagtgcagaaaaaacatgagctacttctttgtcagggtttgcaggtcaaattggtcccaatggcttaggatacatttcaagtgtgagcctgttgatacctgagtgtttcccatctgaaagacaaaactgcccatggttttgggttgtttgttccccccacagcccaagaacctgcaacggtacctggaccctgctgatcggaatagttgggctcactgacacagcagcagatccccctcttgcccaagaacctgcagcagtccctggaacttgctgatcagagtagttgtgctcaccagtgcagcagcagaaatactagttttcctcctagaccacaaggaggaccaaggaaggtcagatttagtggcccttactgacacattctcaaaaacctgcacccttgcctgtcctcatagaacacaaagaggacagagaaaaatcggatatagtggccctgacacattctcgaaaacctgttagagtcctatgcgctctcctgttagtattgggaccttacccctgtcctataaagatggtatgccccaaaactgaagtggagggccatgccctgagggagggaagggatctccagggttagaagagtgacaccttttgtcctcacttctcatcatatgaataggaaggatataatttctgaggctccccatatcctagcttcaggaatagcttttgttaggcctgctagtctgaggatggatcctaaaattccagatagtcccccaccatggggctttgggcaaaaattatgtctttctgattggtgagcccaggtgcctaaagaagggaacagagtcctgaggtttatactagaaattattcttataggagaaactagaaaagcaccagagataggaagtggtttttagaagtgggactagccttggagaagagaggtgagaggaaatttgtctgacaggcgttaggacccaagaggcaagggtcagtatagataggatagatgggtgagtctcgcttggacgacatgactttgagagttctgctcatagcctcagggtcaaccaacttgttgtcaggaccccagagctgaatggctttcctctctgttgacctttggctgagcccggaagtacaggaaaagcagaagcttgttccaggcaaaccaacactcccaactccgaagcattggggtttgttagagagccctttcccaaaaatcctgacacccatgtctttagtccggcagccacgctagttgcttttaattggccaacaggtgcctggtatttagcccccaaattctaaggaaaaatagaacagaatagcaagagaaaggggtccaatggtactcaccgcttagtgataggtgatagtcccttcgtgtttgccaagatgtgtccagagttggttccttctggtgggttcctggtctcactgacttcaagaatgaagccacgggccttcatggtgagtgttacagctcttaaaggtggcacggacccaaagagtcagcagaagcaagatttattgtgaagagtgaaagaacaaagcttccacagtgtggaaggggacccaagcgggttgctgctgctggctggggtggccagcttttattcccttatttgtccccatccacatcctgctgattggtccattttacagagtgctgactggttcattttacagagtgccgattggtgcatttacaatattttagctagacacagagcactgattggtacatttttacagagtgctgattggtgcatttacaatcctctagctagacgcagagcgttgattggtgcatttacaatcctctagctagacagaaaagttctccaagtccccactcgacccaggaagtccagctggcttcatctctcagtggcaggaataatttttgtcctgtcctggttttttttctatgggtgtctcagagaatggtgggattaagggaggactccttttttctttaaattccccagggcaaaccccagatgggtcttccttcttgcttgtttatgccaggaagaactctgaagtttactctgtctactggaacatgcagagtgcagtagaggtttatttgtctatgcactgtaatgatggggttgtgaggtcctcgtcaaacttgtttccattttattcctttaaaaaatgtttcccattccatcatcctgtgtcttgactcctaatagttttatttattggcatgtgtaagttttgcctgggaatatctacacattgcctgggaagtataaaatctccaatgtggcaggtttatccctttgtccctctaagtcactagttggtaatttttctttttattcatatacattttaccagggattatggagtctctttgaccctggcagtgaatcactaaatctggcttacctttttctatacttgggttgatcattattactaaggttttcctgatgttcttcctcttcaggtgggctgatattcagtcagttgacattaataggattgaactctttttttgcagtgaaattcagttgttacttgtctgtgcttctgccttgggggtcattgaatattttgaatatctatgaaatgtctaagattccttgctttaccaatgtctgtggcctcctcattgtctttgggagtgttctaactggactatttatagttgttatatggaccttatgcactaaaataatgacatttatttattctatgaccactctgtttccctgtcatacgttactcagcttctgcttttaactgggtacatattcttaactatatcattatctccagattgtagcattttgttttctacttctttcttcctccccatttccttccttccttccctcctctctttccactcatatggtttagtggcatgtgagaacttcagtgtggttagagactctgccaatggggataaatttcatgtactgtacaatgctgatgtcatctggtttcttgacatatgtcctgtactaactaactctcaatgaaagagataattaatgaagacatgtgttttaacaaagttatataactgcatcaaaagtcacgtgttgtaggtgccccccccatgttttaaagcaattcagaagtacaagttaataaaaattaataaaagagatcaaaataatttacatttttaacatattatttctacactacagagggtatttttaaacatatcttgtggaacccacagccttctttggcaatcattgattcaaagtacaaatcaaaatccctatatgtatgtatccggattactctcacagtaaaatacaaatgtcttatattggtcgtattctgattgaatgactttttaagacccgggtttaaagaggtatagtttacatagagttaaattcactcttttagatgtacagtttgaagagctttgagaaaaatgtaccatcatgtaactaccaatacagaaagctgtataacctttcctttactccaaaatgttccctcctgccccattgtagtcaatccatatctctgtcagttcctgacaaccactgatttgatttctgtccctatagtttttcattttccagaatgtcatacaaatggaatcataaagtatatagccttttacttctggcttctttcacttagcatatttttaaaatattcaccaatgttgttgcatatatcagtaatttattccttaatgtagctgagaggtattctaacatatggatgttctgcaatttgagtatccaattaccagtagatttgtagtttttggcaattatgattaaagttgttaaacattctagaataggtctttgtgtatacataagttttcatttctcttgagtaaatacttaggaatgggatttcttggttttatggtaggtgtgtgcttaactacattagaaattgtcaaactgttttctaaagtagctgtgacattttgcatttccaccagcaatgtatgagagttccagttgatacacatcattgctagaagtcgatactgtctgtattttattttatttttaatattagctattcctttttttaactttgaacctttttttaaaattttattattattatacttttaaggttagagtacatgtgcacaacgtgcaggtttgttacatatgtatacatgtgccatgttggtgtgctgcaaccattaactcgtcatttagcattaggtatatctcctaatgctatccctctcccctccccccaccacacaacagtccccgatgtgtgatgttcccctttctgtgtccatgtgttctcattgttcaattcccacctatgagtgagaacatgcagtgtttggtttttgtccttgcgatagtttgctgagaatgatggtttccagtttcatccatgtccctacaaaggacatgaactcatcattttttatggctgcatagtattccatggtgtatatgtgccacattttctttttttttttttttttttttctgagacggagtctcgctctgtcgcccaggctggagtgcagtggcgtgatctcggctcactgcaagctctgcctcccgggttcacgccattctcctgcctcagcctcccgagtagctgggactacaggcgcctgctaccacgcccggctaattttttgtatttttagtagagacggggtttcaccgtgttagccaggatggtctcgatctcctgacctcgtgatccgcctgcctcggcctcccaaagtgctgggattacaggcgtgagccaccgcgcccggcgccacattttcttaatccagtctatcattgttggacatttaggttggttccaagtctttgctattgtgaatagtgccactataaacatacatgtgcatgtgtctttatagcagcatgatttatagtcctttgggtatatacccagtaatgggatggctgggtcaaatggtatttctagttctagatccctgaggaatcgccacactgacttccacaatggttgaactagttgacagtcccaccaacagtgtaaaagtgttcctatttctccacatcctctccagcacctgttgtttcctgactttttaatgattgcctttctaactggtatgagatggtatctcattgtggttttgatttgcatttctctgatggccagtaatgatgagcattttttcatgtgttttttggctgcataaatgtcttcttttgagaagtgtctgttcatatcattcgccgactttttgatggggttgtttgtttttttcttgtaaatttgtttgagttcattgtagattctggatattagccctttgtcagatgagtagattgcaaaaattttctcccattttgtaggttgcctgttcactctgatggtagtttcttttgctgtacagaagctctttagtttaattagatcccatttgtcaattttggcttttgttgccattgctcttggtgttttagatgtgaagtccttgcccatgcctatgtcctgaatgatattgcctaagttttcttctagggtttttatggttttaggtctaacatgtaagtctttaatccatcttgaattaattttagtataaggtgtaaggaagggatccagtttcagctttctacatatggctagccagttttcccagcactgtttattaaatagggaatcgtttccccattgcttgtttttctcaggtttgtcaaagatcagatagttgtagatatgcggcattatttctgagggctctgttctgttccattggtctatatctctgttttggtaccagtaccatgctgttttggttaccgtagccttgtagtatagtttgaagtcaggtggcgtgatgcctccagctttgttcttttggcttaggattgacttggcgatgcgggctcttttttggttccatatgaactttaaagtagttttttccaattctgtgaagaaagtcattggtagcttgatggggatggcattgaatctataaattaccttgggcagtatggccattttcacgatattgattcttcctacccatgagcatggaatgttcttccatttatttgtgtcctcttttatttcattgagcagtggtttgtagttctccttgaagaggtccttcacatcccttgtaagttggattcctaggtattttattctctttgaagcaattgtgaatgggagttcactcatgatttggctctctgtctgttattggtgtataagaatgcttgtgatttttgtacattgattttgtatcctgagatttgctgaagttgcttatcagcttgaggagattttgggctgagacgatggggttttctagatatacaatcatgtcatctgcaaacagggacaatttgacttcctcttttcctaattgaatactctttatttccttctcctgcctgattgccctggccagaacttccaacactatgttgaataagagtggtgagagagggcatccctgtcttgtgcctgttttcaaagggaatgcttccagtttttgcccattcagtatgatattggctgtgggtttgtcataaatagctcttattattttgagatacgtcccatcagtacctaatttattgagagtttttagcatgaagggttgttgaattttgtcaaaggccttttctgcatctattgagataatcatgtggtttttgtctttggttctgtttatatgttggattacatttattgatttgtgtatgttgaaccattcttgcatcccagagatgaagcccactctatcatggtggataagctttttcatgggctgctggatccgatttgccagtatttttttgaggatttttgcatcaatgttcatcaaggatattggtctaaaattgtcttttttggttgtgtctctgccagtctttggtatcaggatgatgctggcctcataaaatgagttagggaggattccctctttttctattgattggaatagtttcagaaggaatggtaccacctactttttgtacctctgttagaatttggctgtgaatccatcaggtcctggactttttttggttggtaaactattgattattgcctcaatttcagctcctgttattggtctattcagagattcaacttcttcctggtttactcttggggggatgtatgtgtcgaggaatttatccatttcttctagattttctagtttatttgcatagaggtgtttatagtattctctgatggtagtttgtatttctgtgggatgggtggtgatatcccctttatcattttttattgcatctatttgattcttctctcttttcttctttattagtcttggtagcggtctatcaattttgttgatcttttcaaaaaaccagctcctggattcattgattttttgaagggttttttgtgtctctatttccttcagttctgctctgatcttagttatttcttgccttctgctagcttttgaatgtgtttgctcttgcttttctagttcttttaattgtgatgttaggttgtcaattttagatctttcctgctttctcttgtgggcatttagtgctataaatttccctctacaccctgctttgaatgtgtcccagagattctggtatgttgtgtctttgttctcgttggtttcaaagaacatctttatttctgccttcatttcattatgtacccagtagtcattcaggagcaggttgttcagtttccatgtagttgagtggttttgagtgagtttcttaatcctgagttctagtttgattgcactgtggtctgagagacagtttgttataatttctgttcttttccatttgctgaggagtgctttacttccaactatgtggtcaattttggagtgggtgtgatgtggtgctgaatagaatgtatattttgttgatttagggtggagagttctgtagatgtctattaggtccgcttggtgcagagctgagttcaattcctgggtattgttaactttctgtctcattgatctgtctaatgttgagagtggggtgttaaagtctcccattattattgtgtgggagtctaagtctcttcgtaggtcactaaggacttgctttatgaatctgggtgctcctgtattgggtgcatatatatttaggatagttagctcttcttgttgaattgatccctttaccattatgtaatggccttctttgtctcttttgatctttgttggtttaaagtctgttttatccgagactaggattgcaatccctgcctttttttgttttccatttgctttgtagatcttcctccatccctttattttgagcctatgtgtgtctctgcacgtgagatgggcttcctgaatacagcacactgatggatcttgactctttatccaatttgccagtctgtgtcttttaattggagcatttagcccatttacatttaaagttaatattgttatgtgtgaatttgatcctgtcattatgatgttagctggttattttgctcgttagttgatgtagtttcttcctagccttgatgttctttacaatttggcatgtttttgcagtgcctggtactggttgttcctttccatgtttagtgcttcctttaggagctcttttacggcaggcctggtggtgacaaaatctcccagcatttgcttatctgtaaagtattttatttctacttcacttgtgaagcttagtttggctggatatgagattctgggttgaaaattcttttctttaagaatgttgaatattcgcccccactctcttctggcttgtagagtttctgccaagagatcagctgttagtctgatgggcttccctttgtgggtaacctcacctttctttctggctgcccttaacatttttttccttcatttcaactttggttaatctgacaattatgtgtcttggagttgctcttcttgaggagtatctttgtggcattctctgtatttcctgaatttgaatgttggcctgccttgctagattggggaagttctcctggataatatcctgcagagtgttttccaacttggttccattctccccgtcactttcaggtacaccaatcagacatagatttggtcttttcacatagtcccatatttcttgcagtctttgttcatttcttttcattcttttttctctaaatttctgttctcgcttcatttcattcatttcgtcttccatcgctgataccctttcttccagttgatcacatcggttactgaggcttctgcatttgtcatgtagctctcgtgccttggttttccgctccatcaggtcctttaaggacttctctgcattggttattctagttatacattcgtctaattttttttcaaagcttttaacttctttgccatcggtttgaatttcctcctgtagctcggagtagtttgatcatctgaagccttcttctctcagcttgtcaaagtcattctccgtccagctttgttccattgctgttgaggagctgtgttccttcggaggaggagaggcactctgatttttagagtttccagtttttctgctcttttttttccccatctttgtggttttatctacctttggtctttgatgatggtgacgtacagatggatttttggtgtggatgtcctttctctttgttagttttccttctaacagacaggaccctcagctgcaggtctgttggagtttgctagaggtccactccagaccctgtttgcctgggtatcagcagcggtggctgcagaacagcggatattggtgaactgcagatgctgctgcctgattgttcctctggaagttttatctcagagcagtacccggccgtatgaggtgtcagtctgcccctactggggggtgcctcccaattaggctactcgggggtcagggacccacttgaggaggaagtctgcccgttctcagatctcatgctgggagaaccactactctcttcaaagctgtcagagaggggcatttgtctcattctggttttacttgcatatcctcaattactaatgctgttgagcatattttcatgtgttcacttgctattcatatgtcttttctgatgaggtgtctgtttaaattcactatttttatttggttattcaataataatcttattattgaattttgagagttctttatatattttggataaaagtccttcatcacataggtgttttgcaagtattgtctcccaatctgggttttcttttcatgttattttttgaaaagaacttgttttcaagtccaatttattaattttttatggtccatgctttttaaatcctaataaatctttgcctagaccaaaatcttaaagattttctacttttttttacaagttataatttttggttttatatttaggtcgctaacccattttgagtaattttgtatatcttcttagctgaggggtcaagaagaatattttggtgtgtgatgtccagttgttccgtaccatttgttgaaaagaccgccatttcttcatttaattacctttgtccaaaatctgttggctatatttgatggtctctttgcattctattgatctataggtcaatcctatgtcaataagaaactgtcttaattaccatagccttgaaatcaggtagtatgaatcttctaacttttgtatttttttctcaaaatttgtctggctgttctggttcctttgctctttccacatacattttggaattagcttctcaatttctatgaaaaacctactgggattttgattgagattgcaataattctacagaaatgttggggagatataacatcctaaaaattagtatttcagttattcagtcaaagaacatggtatatttctctatttatttaggtcttatttaatttatctcatcagtgtgttgaagtcttcagcattcaaatcttgtacatagttatcctcccttagtatttcccagtttttgatgatattacaaatggtactctttaaaacttgaaattccaaattttattgtgtagaaataaaataaattattttatattgactttgtgtctggcattattattaaacttacttattaggtctcatagcttcatagcttttttttggtagctccattgggattttctacatacatcatcttgtctgtaaatgcaattttacttcttcccttccaatccgtacataatttatttatttttcaggccttattgtaattggctgggatcgccaatgtaatgctaaataaaagtggtgaaagcaaacatccttgccttgtttctgattttaggggaaaagcattctatgttttaccatatgatgttaatcataggctttttattttttgtttttttgtagatgtctttaatcaggttgaggaagatccattaaactcttactatgctaaatgtcttttatcattatttgtggaatgttgttaatgctttttctgtaaaaattgagatgattttttttctccattttagtctgttgatatggtgaataatattgattgattgatttgcaaatgttgaaccatccatacattcccaggataaactccatttggtcataatgtattattctttttccttatcatcagctttgattaatagcatttcgttaaggacttctttgtctatgtttatgagagatattgatctgaagttttctttatttgttattttctagtttgggtatcaggttaatgctggtattataaaatatgttgaagagtttttcctcctttcctcacataattgtcatctggcttgcataacttatattatttgtgcttcagttggagcctggagtgctacaagatttttttcaacgattttccactctaaattttcagtagtctcttcatacttgtgccaccagcagagagtctctcttcttactcttgttcacatgacagcagttgactgctgttattatcaggtgctaggctcatggtgaggtaggaagtactgtctgttgtcctgtctagccacagccttagcggggctctatgaacatgaacctttagggcaaggctttctcatcattctttcctctccccatggcagctaacctctgatttgtatatgtagttggtctttggtcagatatattttcttgcctctttctcaggctgcttttcattcatgtaagaactgggttagagagggttccctccagggcagaggtttttcccttctacttctccctgagcagcaatggatctctgcctttgtcctggaaattagggggtttcctatctctccctcccaggcaagtaagttttgcttctacccttccaccagaagcaagggatttttgcctgggttcaggagagggtttgctgcttctcctctagtgtcttaaggctttgcttctcatgacagaaaggttctgtggggttgtatgtctgtcccccagcaacagctgattaccttctgcataccagaaccagtgacatggactcttttaggtctcctgctttgtcctgagtcatttttctgagcttctggttaaagactgtgaaaaagaacttggcattaagtgtgtacgtgttggggttccagatattccaaactgacaaagctaacgcatgctttacttttaaaagttcattaaaatttcagcttgtatgacagtcacttctgtctccatgcttggacagaggttcatatgttttgtctttctttaaaggggctgattaccctttggaattcactttaactcagttccctgataggctcaaggaaagttatgatttcatatgttatctagagatttatcattattaagttatgaatgacattctcttacagctttttatatcctaagtggaagtgtaactggtgcttttgaaaaaatatacgtatgggtcttatttggggaatttttgttattaagtatgctttaggaagaagcagatgctttctaaggcatacttgcaaagacttgctttttatataatggttattactacattttttcatttaaatttttattatttgaattgctatacattaaaatcatactttaaagtataatacactatacttgaccattttttgtatacggttctctggattttaacatatataaagattcatataaccaccaccacagtcagaaaagtttcatcaccctcacaacttccttggtttgtccttttatagccacacccttccccacctgtaacacctggcaacactgatcttttcaccacagctatacttttgtctttttgaaaatgtcatataaatgaaatcacaaggtattgtaaacacaccctagatgacccaccccttatataatccccatggtaagaaaacacttttggcttctatcatcattgtcattattaataataatactaatgttgttatacagagtgagttggttatggccctctcttccaggaaataaccatctaattattaaataagatcatatgtcaagtgcttgagaacattgcctggcacatagaaaacttgtgtaggtattagatattattatatctgtaatgttatctatctttatcataaaaatattaatattgcagtattagcatttgctattattagttatcctttttgtccacctccttctacaaattggtgaaatagaaaatattgttcatggaattaatcttttcctttgcatttcattttgaaaatggtgccatagtatagcatttcctgatttatgtgcaatctagaaaatatccaaaataaagcaaaaaaaattttaacataattatcatactctgtgggcatttgtttttttccaaacatgtgaaatgaaggcagaatgaaaaatactgcattataaaatatataataaaataaatctcttcatttaaataaagccatatatatattggcctgataatcctctttagattatgtattaataccttgaaagcattgaaaggattaattgtataataagaattttgttattttcattgtaatttttttttttttttttgagacagagtctcgctttgtcgcccaggctggagtgcagtggtgctatcttggctcactgcaagctctgcctcctgggttcatgccattctcctgcctcagcctcccaagtagctgggactacaggcacccgccaccaagcctggctaattttttctttttttgtatttttagtagagacggggtttcaccgtgttagccagaatggtcttgatctcctgacctcgtgatccacctgtctcggcctcccaaagtgctgggattacaggcgtgagccactgagcccatccttttcattgtaatttgtataagatcaactattttgttctagtactagaaaaaaagactggaaaacagagaggctttggtaagatagaagtttaatgttccactaacttaaagagattggaatgaggtgatccccattattgagtggtttagggtcacacgtccctagtgagttgcatccttttccgaagtcactttgtggtccagaaaggctgcctgaactctctatcatgatcaaattctggaaagcaagagaaagggggaaagtggaaaaaaaaagcccaagctggttatttatccccctttagagatgccttctctagaatcttcacagcaatttctgcttgcattctttggcaagaaattccatcacatgaccatggcttgcagtgagggagtctgggaaagacaatattttaggagattaaactgccaccttcaatacaactggtgttctgctaatgaggaagaatgcaaagagtggatcttgggtgagcaactagaagtttctgtctcattcattacaaatatgatatacgattgtaaaaaaaaattaaaccagtaacagaagtctataaaagattgtaagggaaaatttcctgtgacagagagaacgctacatgtttgtcaacctctgtctcatttttctcctggggtcacagggagattacattacccagcctctatgccagttggggatccatgcctaaattctggttagtggactgttaactagcagtgatgtttgccagacctgaccactaaaaccttccatatggctctccatcctccctcttgtttaggctgttagctagatgcaaagaatgtaatgaaggtctttaagacccaagtagatggtagagccacaggatggaagaagattgggtctcttaatgactgcttagaacagagaaacttgcccctatttggccacgtgatgccttacgttgaaccttgacatcagcaaaataaacttttgggggattgaacctctgagatttaggggttgtttatagcacacagcattatatactctaattattagagtccccagtaatacaccagcctagagatggctttcattgatttattggtatttttattttaatattaatgcttatataaaggtgaatgttaaaattgtaattatatatatatgagtaatactacatattatatgatacagaagccaaataaaatactttattccccaaatcatcactagtgataattacaattgactttctgagttaaatcagaaatctatacttaaatttaacatagaattttgatgaacagaatgtacaaagtatatttcggacctgtaaataaagacataaatttttaaaaattaattcccctcacattgaatcacccaagtgaaaacaacaaaacataagaaacagaattgccggccgggcgcggtggctcacgcctgtaatcccagcactttggaaggccgaggcaggcggatcatgaggtcaggagatcgagatcatcctggctaacacggtgaaaccccttctctactaaaaatgcaaaaaattagccaggcatggtggcgggcgcctgtagtcccagctactcggaaggctgaggcaggagaatggcgtgaacccgggaggcggagcttgcagtgagccgagatcacgccactgcactccagcctgggcgacagagtaagactccgtctcaaaaaaaaaaataaaataaaataaaaaataaaaataaaaaaagaaacaattgttaattatcgatgagggcatatgaaatgagtgctttcctggtaagccaatgattttaacatctagcaagaaataatcacatatgtgaagaaatctgtgaaactttcctgaagggttttagagaagaactgaataaaagtagagacgtaccatgtcctggaaacagaaagaaaaacaaaagaattataaagaaataaagtctccctcaaataatgtacagtttttgtgtaatccaaatcaatatccttagcggtctattttaatggacttgaccagctgggtttaaaattcacctatggggttaagatgccagagatggcaagaaaaaaatatgtaaggaatattagtaactgtgttttgattgaaatatgataaagtgatttgctacctcttttcttcaagaaatcacacaccctaaggttttgaatagaaattaagcacacttgttgacatttcacttaaaagttttttaaaaaggaacattactgcagttcaacacttcagcggtttctcttggcaattccccctcccctgatccaggcaggggctttctgttcttcatacagccagggggcggaccctcctctgagcctctaggaaaggactgtgcctggccagtcttgggggaggagctgcatttcaccaggctgcagctgacaagaagcttcaaggtaatggctgtctcaaagctacttattttctagtgacttctcaggttcttagagtaaagataggaaaaatagggatggcagtcttgcagtggtgggaaaggtgtgtggttttgagaattgctggcattttgaaggagttaatcctctggaatttttgtatcttccaatgctttagaagtccacagtgggagaaggggtgatcagattacagcctctaaattaaggcttcagtgcagcatgactgttcctgctaatttattgagttggcatcccctgtttatattgttagacactccgttattgtttgaatatttaggcatttattaatttgcacagaaaaataacttagaagtatttgacagtatggggggaaatacacctataagttgattgatacataaaaagactgtatgtgtcacatgtacatgagcatggaaaagggatcaaatatgtagttgagtgaagatacttgcacataatatatgcatgtatatatgtgatttttaaaatctggaggaaaataatctctcatgttaacagcatttctatgtggccaggtcaccattttaatattattttttaaactatttcagcatctttagagtagattactcagtgattatttttataatcatgaaataaaaaggaagctgaaacagcatacccattttgtttcaaatacataaacacacatagacacacaaagacatgctcccatatcccccctgtcttctgccaatagtggaaattttcctggacttgatcaagtgggtagcatagttgccgtgtcaactacaggcctgcatgtgtcctccctgcagtttattctttcagatcacagataccagcatggtcaggaagagccatgtttctgtatcatctaagtttaagagtgagaaacagaggatttggtttatttctatacgcagttttcaaaatattatatggcatggtacatctgtacaatgaaatactatgcatttggcaaatgcctgagaaacagtataaggtgaaaaaaattatataatagaacatactatatgttctcacttaagaataaatatgtatgttagtatacctatgtatgttactacatacaaatcaggaaaaacatatctatgaaagttttaacaggattacttgcatagttgtagagacagggatagctttaccattttctactttttaaaattattaatatatatttatctggggtttgttacatcttcggggaaatattgagataaatgctacaggcttgggggaaaacctcatttaaagtaatttatatcaaattatgtggaaaatatttaggtgaatgatgttcaagctatgctcttgagtggaatctgaagctggtttcaaattttcaaatgagatacatttatctttatatgtaaaaaaacttctagaaatacatatagtaaatattgatagtggttatttgtctagagttgtactttattttacatatacctttttgcatcttctcaatttttaataagcttctggatgttttcataatcagaaacactgtagttgaagacattatgatctggttagaatcccacacaacaaaacctttaaatttaacatagaaaattctagaagcctgagaagactcctggctagctgtatgtagtccttatggaataaggatggaaactacatctgatagtgagtgaagtccccttcaactcatgagcagttctggttgtcatcataagagggcactagacagattttctaaattttttccagccaaccattgttatgaagtagacaaacaaagcaatgaatctattttgtattagtaaactatgagacagcatgtaattgcaattccattgaaaaaaaattgtatgtgtaaagaagaaaaattccacatgataactgatgtgagcattcaaacaaaatctacattctgactcatagtatacacaagacaattgttacttcttttttgcctatctccactccaccatactttttttcagagatgatttcaacataaacaaaatagcataaatttatcccagagagcatttcaaattataaaaataagaaggaaaataaacatggcatctacatcacggatgaaattttgcctcattcctatgttttagaatgacatgcctgttaaagtttaggtagggaaaattcttgacacttttcccatttcgttggttttattccctgccttccttttgaacagcataggttaatatcagagaagcacatgacctgggcaatctgcatgtgatgccaaccaagccttggaaagacagtgcaaccacagaagtgccgagctgggtaaaggccctgctggacaagcagttggagtgagaatcaagacagccggaccacaggaccagacccaccaggatccacatgacagtgggtaagactcttcccatttcataaaacatttcacggtagaaacatgttgagaacttggagaataatgtattttgcctcctgctctatagccatcctagcattttaaattaaaagccatatttcatgttagaatcatgttaggttgatgatttggggtacaattcattttgccccctgttctgcagccatgacactttataattaaaagtcgtaacttctagtttggagaagaatctcactttttaaacgattataaagaaatcatgcaagaggctattcaggtgtctccctgaggttcaggccaatggcctgactctccaatcctttataatgatcctatccaagaagttgtcatagcagtaattctgaatctctgccttctaaaattatgcttttagaagagtcattgtatttttgtttgggtccaggagatacagaccctattgtctttgtctgcctgctccaggtgaacagtggagcacttgcagcttaaaaaatacaaagctgtgttttcaactagaacattgctcctcaaagagttctttttaaacatgtagcattagaggcacttgagaactttttagaaatacaaacttttgagcacaagccaaaccttctgaataaaactctcatgatggggcctagaaatataagtccttcagatgattctgacacatgttaagtttggagacccactgctataagtatgacattagggacacaaaagtaccaaagtatgtaattacatagagttcctgacatcccatcacaagctctcccgcctctgctgccatgtacatacattcactccttagtaagcatgttcaataatgagggcaagttcttcaatctgaggtattataaaacaacttttctggtttttttccacccctcccccccgcaatcttatattatgctgcctcctctgtctcatgccccactgcacattgttcttttatcccttctccatttgtaattgctcacctcccgttttggcgaaataatcgtatttgctgatacttttcttcttttccttttccctgtttcagtatcttccggtatcatcctattcaaacaacgtgttcaagcatccttggaaaattgaggtggagaacaggccacacaagctgtaaaccctttgtagtcataagacgaaagaggatttgttaagagtattgttttgggtagaacttgcattagcagtgacaaagatagccacctcaacctttggtaataacccttggcacacaaatataaaccataatggagtctgagtatgtcctatgcaactggaaagaccagttgtggccagcaaaagttttgtccagatctgaaacttcatcaaacagtaagaggaaaaaggcattttctctagaagttcaaatactctcactagatgaaaaaattaaattggacagcacagaaacaaagatcctaaataaatctcaaattgaagccattgctgcctcattaggactacagtcagaggacagtgctccacctacagaggaaactgcctatggaagatcactaaaagtggcactgggtattctgaatgagagaacaaatttgagtcaagcaagcacttcagatgaagaggagatcactatgctgtctcaaaatgtaccacaaaaacagtccgattcaccccctcataaaaaataccggaaggatgaaggtgacttaccagggtgtcttgaggaaagggaaaactcagcatgcttgttagcatcttcagagagtgatgattccctgtatgatgataaatcacaagcacccacaatggtcgatactattccaagtgaagtggaaacaaagtcattacaaaactctagctggtgcgagactttcccttcactttcggaagataatgatgaaaaagagaacaagaataagattgatatctcagcagttatgtctgtgcattctgcagtcaaagaggaaagtgcatgtgttaaagatgaaaagtttgctccacctttgtcacctttgtcatcagatatgctcattatgcccaaagctttgaaagaagagagcgaggatacctgcctagagaccctggctgttccctctgaatgctctgctttctcagagaatattgaggatcctggagagggtccctcaaatccatgcttagataccagccagaatcaaccttccatggaatcagagatgggggctgcagcatgccctgggagttgttcaagggaatgcgaggtttcatttagtgcctctaaccctgtctgggattattcacatcttatgagtagtgaaagaaattttcagagactggattttgaagaacttgaggaagaaggtcaagcctctgacaagtcattgcttccaagtcgcattaatctttctctattagatgatgatgaggaagacgaagaacttccacgcttcattttacattatgagacacatccgtttgaaacaggaatgatagtctggtttaaatatcagaaatatccattttggccagcagtgataaaaagtatcagacgaaaagagaggaaagcaagtgtgctttttgttgaggcaaacatgaattctgaaaagaagggcattagagtaaattttagaagattaaagaaatttgattgtaaagagaaacaaatgctagtggacaaagccagggaggattatagtgagagtattgactggtgcatctcactaatttgtgactacagagttagaataggttgtggttctttcacgggctctttgcttgagtattatgctgctgatattagttacccagttaggaaagaaacaaaacaggatactttcaggaacaaatttccaaagctgcataatgaagatgccagggaaccgatggctgtaacttcccagaccaagaaaatgtccttccaaaaaattctccctgaccggatgaaggctgctcgggaccgagccaacaagaacctggtggacttcattgtgaatgcaaagggaacagagaaccatcttctggccattgtaaatggcacaaaaggatccagatggctgaaatcatttttgaatgcaaataggttcacaccctgtattgaaacatactttgaggatgaagatcagttggatgaagtggtgaaatatttacaagaagtctgcaatcaaatagatcaaataatgccaacttggataaaagatgataaaattaaatttatcctagaagttcttctgccagaagcaattatttgttcaatttctgctgttgatgggttagattacgaggcagctgaagcaaagtatctaaaaggaccatgtctaggctacagggaaagagaattatttgatgcaaaaataatatatgaaaagagacgaaaagcaccaacaaatgaagctcactaaatgtgctgaaagttgaaaccatgacagggagctctcatagatactagttgaaaaaagtctctgaacaattctctctaatacatattttctgcaaatgggagcatggataatgtgttcacttttttttgagatctctaggatctgtggttataattacatctttatttccttttcttgcgcttcttcaaagttaattttgtcaacatatttcagcagttctactttcccgtacattttttagaaagcataattcctaaatgattttgaagggaaagtactattttgttttatgacacttttgagtctatgctgtattgttaaatatattgtgcacaattattttaatgttaaaattgcactggtgttagatattaacgaagatgattggaaaaaagtctgaaagatacatcatttctatattccttgcttaatttttataaaatattgggttttcccttaagatagttgaagtatttttcttgccatgcctatttatttttgcaaaaaaattatctgctatatagaaccaaagatagattcgctttgctatatattataactatacttgcttttgcaataaaacaacttgtaattcaaaaatgaagattatctgcatatagtatttatgtgtgtgaataacatcttggcatgcagaaaataattttggctgctttgaacaggattgttcattctacacttccctgaaattccatttttgggagtgtctatccttcaatggctgaatagttccttaaatcagacaagctcctggctatcttcataaccagcagtgagtagatcttcttaatatagctgtctgaccacatgtggaaggcagctagacttattgtatttacagcctggccactctagacatatgaatccaggctgactgcttaagtgcccatctcgtttgatataaactgtagaaaaggcaagcactttgttctgagtcttttatctccaaatacatagttgtctcaaaccaagcaagctttatcaaaatggcttatttgcagaatagttccaatgatataaatgccaactggcaagtcattccaaactgcttgaaggagtagatgaaccagaatctgagaatttggaaataggtcacagaaaaagcctccatttggctaaatatgacaattatctgagtatggttaaatacatacaattaaatgtctgaagccaatgagttgtttattctaacttgaaatatatttttgtgagaataaatgttagaaaagttagtttattttggaaacctgccgtgaaaggaaattctaaaggcttaacatgaaactgtctgcatttctacctgttagacaatggttgctcttgggctcttgttatacagtcatatggctacacgtttatgtaaagtttgcaaaagtttggtgttttgatttataataaaactgttatattattcaccttg

SEQ ID NO: 2 - DNA, 2091 bp, synthetic construct
  source 1..2091: mol_type = other DNA
atggagtctgagtatgtcctatgcaactggaaagaccagttgtggccagcaaaagttttgtccagatctgaaacttcatcaaacagtaagaggaaaaaggcattttctctagaagttcaaatactctcactagatgaaaaaattaaattggacagcacagaaacaaagatcctaaataaatctcaaattgaagccattgctgcctcattaggactacagtcagaggacagtgctccacctacagaggaaactgcctatggaagatcactaaaagtggcactgggtattctgaatgagagaacaaatttgagtcaagcaagcacttcagatgaagaggagatcactatgctgtctcaaaatgtaccacaaaaacagtccgattcaccccctcataaaaaataccggaaggatgaaggtgacttaccagggtgtcttgaggaaagggaaaactcagcatgcttgttagcatcttcagagagtgatgattccctgtatgatgataaatcacaagcacccacaatggtcgatactattccaagtgaagtggaaacaaagtcattacaaaactctagctggtgcgagactttcccttcactttcggaagataatgatgaaaaagagaacaagaataagattgatatctcagcagttatgtctgtgcattctgcagtcaaagaggaaagtgcatgtgttaaagatgaaaagtttgctccacctttgtcacctttgtcatcagatatgctcattatgcccaaagctttgaaagaagagagcgaggatacctgcctagagaccctggctgttccctctgaatgctctgctttctcagagaatattgaggatcctggagagggtccctcaaatccatgcttagataccagccagaatcaaccttccatggaatcagagatgggggctgcagcatgccctgggagttgttcaagggaatgcgaggtttcatttagtgcctctaaccctgtctgggattattcacatcttatgagtagtgaaagaaattttcagagactggattttgaagaacttgaggaagaaggtcaagcctctgacaagtcattgcttccaagtcgcattaatctttctctattagatgatgatgaggaagacgaagaacttccacgcttcattttacattatgagacacatccgtttgaaacaggaatgatagtctggtttaaatatcagaaatatccattttggccagcagtgataaaaagtatcagacgaaaagagaggaaagcaagtgtgctttttgttgaggcaaacatgaattctgaaaagaagggcattagagtaaattttagaagattaaagaaatttgattgtaaagagaaacaaatgctagtggacaaagccagggaggattatagtgagagtattgactggtgcatctcactaatttgtgactacagagttagaataggttgtggttctttcacgggctctttgcttgagtattatgctgctgatattagttacccagttaggaaagaaacaaaacaggatactttcaggaacaaatttccaaagctgcataatgaagatgccagggaaccgatggctgtaacttcccagaccaagaaaatgtccttccaaaaaattctccctgaccggatgaaggctgctcgggaccgagccaacaagaacctggtggacttcattgtgaatgcaaagggaacagagaaccatcttctggccattgtaaatggcacaaaaggatccagatggctgaaatcatttttgaatgcaaataggttcacaccctgtattgaaacatactttgaggatgaagatcagttggatgaagtggtgaaatatttacaagaagtctgcaatcaaatagatcaaataatgccaacttggataaaagatgataaaattaaatttatcctagaagttcttctgccagaagcaattatttgttcaatttctgctgttgatgggttagattacgaggcagctgaagcaaagtatctaaaaggaccatgtctaggctacagggaaagagaattatttgatgcaaaaataatatatgaaaagagacgaaaagcaccaacaaatgaagctcactaa

SEQ ID NO: 3 - protein, 696 aa, synthetic construct
MESEYVLCNWKDQLWPAKVLSRSETSSNSKRKKAFSLEVQILSLDEKIKLDSTETKILNKSQIEAIAASLGLQSEDSAPPTEETAYGRSLKVALGILNERTNLSQASTSDEEEITMLSQNVPQKQSDSPPHKKYRKDEGDLPGCLEERENSACLLASSESDDSLYDDKSQAPTMVDTIPSEVETKSLQNSSWCETFPSLSEDNDEKENKNKIDISAVMSVHSAVKEESACVKDEKFAPPLSPLSSDMLIMPKALKEESEDTCLETLAVPSECSAFSENIEDPGEGPSNPCLDTSQNQPSMESEMGAAACPGSCSRECEVSFSASNPVWDYSHLMSSERNFQRLDFEELEEEGQASDKSLLPSRINLSLLDDDEEDEELPRFILHYETHPFETGMIVWFKYQKYPFWPAVIKSIRRKERKASVLFVEANMNSEKKGIRVNFRRLKKFDCKEKQMLVDKAREDYSESIDWCISLICDYRVRIGCGSFTGSLLEYYAADISYPVRKETKQDTFRNKFPKLHNEDAREPMAVTSQTKKMSFQKILPDRMKAARDRANKNLVDFIVNAKGTENHLLAIVNGTKGSRWLKSFLNANRFTPCIETYFEDEDQLDEVVKYLQEVCNQIDQIMPTWIKDDKIKFILEVLLPEAIICSISAVDGLDYEAAEAKYLKGPCLGYRERELFDAKIIYEKRRKAPTNEAH

SEQ ID NO: 4 - DNA, 20 bp, synthetic construct
  source 1..20: mol_type = other DNA
acccccacatatgatcctca

SEQ ID NO: 5 - DNA, 20 bp, synthetic construct
  source 1..20: mol_type = other DNA
ccatttgatgacctattcaa